(19) United States
(12) Patent Application Publication
(10) Pub. No.: US 2007/0065504 A1
(43) Pub. Date: Mar. 22, 2007

(54) Products and methods for treating vaginal infections

(76) Inventors: Shun Y. Lin, Plainsboro, NJ (US); Maria Kang, Hillsborough, NJ (US); Brinda Wiita, Princeton, NJ (US); Kalpana Patel, Princeton Junction, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/395,092

(22) Filed: Mar. 31, 2006

Related U.S. Application Data
(63) Continuation-in-part of application No. 11/080,914, filed on Mar. 15, 2005.
(60) Provisional application No. 60/555,478, filed on Mar. 23, 2004.

(51) Int. Cl. A61K 31/4164; A61K 9/64

(52) U.S. Cl. 424/456; 514/396

(57) ABSTRACT

The methods of the invention treat vaginal fungal and yeast infections. More particularly, according to the methods of the invention, solid dosage forms may be administered to patients to treat vaginal fungal and yeast infections subsequent to awakening at any time during the day more than thirty minutes prior to retiring to sleep for the night.

### FIELD OF THE INVENTION

This invention relates to unique methods of administering vaginal antifungal and antibacterial compositions intended for a single-dose, or multiple dose application.

Infections in the vagina may be caused by yeast (which is a fungus), called Candida and/or bacteria, most commonly bacterial vaginosis. If these infections are not treated properly, the infections can be very uncomfortable and even painful. Conventionally, these infections are treated locally by creams, suppositories, soft gelatin capsules, vaginal tablets and ointments, which contain antifungals or antibacterials. Treatments can last from seven days to one day.

### BACKGROUND OF THE INVENTION

Antifungal or antibacterial creams, suppositories and tablets, in the prior art, are available with treatment regimens lasting for seven days, three days or one day. Reapplication may be required every day. Repeated dosing is very inconvenient and often messy for consumers. There is a consumer preference for one day or single dose application treatments. Furthermore, due to the action of gravity upon the compositions used to treat vaginal infections, causing them to leak out of the vagina when the patient is standing, these treatments have been required to be administered at night just before bedtime. In this way, the patient maintains the compositions in residence in the vagina for sufficient time to effect action against the infective agent.

Applying medication for vaginal infections in this manner is extremely inconvenient for the patient. Moreover, such an application method forces the patient to wait until night-time to begin therapy against the infection, even if the patient is suffering with irritation, pain and itching early in the day. This can be exceedingly uncomfortable for the patient, particularly in the case of fungal infections, which cause substantial discomfort. Thus, there exists a need for a method of applying vaginal compositions more quickly and conveniently to the patient.

### SUMMARY OF THE INVENTION

This invention relates to antifungal and antibacterial ointments for multiple or single application (preferably in seven doses, three day doses or most preferably single doses) to the vagina (also referred to as “vaginal cavity”). The problems seen in the antifungal and antibacterial treatments in the prior art are numerous. Many treatments require multi day treatment and multiple applications per day.

Since creams and ointments may leak from the vagina or the antifungal or antibacterial agents may leach out of the vagina, a reapplication of the cream or ointment is required. Reapplication is required to insure and maintain a certain minimum concentration of antifungal or antibacterial at the site of infection, and thus is very inconvenient for the consumer.

In accordance with the present invention, a method for treating vaginal fungal and vaginal bacterial infections includes inserting in the vaginal cavity of a mammalian species, including humans, at any time during the day or night, a therapeutic amount of the antifungal or antibacterial in a solid dosage form such as an ovule, and allowing the ovule to melt in the vaginal cavity and adhere to the vaginal membrane. Surprisingly, we have found that inserting the solid dosage form during the day, when the patient is moving or even exercising vigorously, the solid dosage form melts and quickly spreads through the affected areas of the vagina without appreciable leakage. As used herein, the term “solid dosage form” means a dosage form that is applied intravaginally and does not flow perceptibly under moderate stress. Such solid dosage forms can take the form of a soft gelatin capsule or ovule containing active ingredient in semisolid form or a suppository, which is defined as a small plug of medication designed to melt at body temperature within a body cavity other than the mouth, especially the rectum or vagina. As used herein, “clinical cure” means that no symptoms of vulvovaginal candidiasis or bacterial vaginosis were detected upon physical examination. “Microbiological cure” means that a culture for candidiasis or bacterial vaginosis was negative. “Therapeutic cure” means that no additional treatment was indicated for vulvovaginal candidiasis or bacterial vaginosis.

An embodiment of the invention comprises a solid dosage form comprising one or more antifungal active ingredients. The antifungal is preferably an imidazole derivative, more preferably miconazole nitrate, clotrimazole, econazole, saperconazole, terconazole, fenticonazole, sertaconazole, posaconazole, itraconazole, ketoconazole, butaconazole, tioconazole, fluconazole, cyclopirox, their pharmaceutically acceptable salts, or a combination thereof, and most preferably miconazole nitrate.

An embodiment of the invention comprises a solid dosage form comprising one or more antibacterials, one or more water insoluble components and one or more water soluble components. The antibacterial is preferably metronidazole, secnidazole, omidazole, tinidazole, clindamycin, sodium polystyrene sulfate, and sodium cellulose sulfate, and most preferably metronidazole.

Another embodiment of the invention comprises a solid dosage form for vaginal use comprising one or more antifungals, one or more water insoluble components, one or more water soluble components, and one or more probiotics. The probiotic is preferably probiotic organism, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

Another embodiment of the invention comprises a solid dosage form for vaginal use comprising one or more antibacterial, one or more water insoluble components, one or more water soluble components, and one or more probiotics. The probiotic is preferably probiotic organism, including but not limited to Lactobacillus and Bifidobacterium species, preferably L. rhamnosus, L. acidophilus. L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

Another embodiment of the invention comprises a solid dosage form for vaginal use comprising one or more antivirals, one or more water insoluble components, one or more water soluble components, and one or more. probiotics. The probiotic is preferably probiotic organism, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

Another embodiment of the invention is the method of treating a fungal infection of the vaginal cavity. A solid dosage form, which comprises an antifungal, and a combination of water soluble and water insoluble components, is applied to the vaginal cavity, preferably only once, and is retained in the vaginal cavity. At least part, and preferably all of, the solid dosage form is melted, preferably on contact with the body and most preferably from body heat. The formulation is spread substantially uniformly in the body cavity. The solid dosage form may comprise an antibacterial in addition to an antifungal.

Another embodiment of the invention comprises a solid dosage form comprising one or more antifungals, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Another embodiment of the invention comprises an solid dosage form comprising one or more antivirals, one or more water insoluble components, and one or more water soluble components. The antiviral may preferably include but is not limited to immunomodulators, more preferably imiquimod, its derivatives, podofilox, podophyllin, interferon alpha, reticulos, and cidbfovir.

Another embodiment of the invention comprises a solid dosage form comprising one or more antibacterials, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Another embodiment of the invention is the method of treating a bacterial infection of a body cavity. A solid dosage form, which comprises an antibacterial, a combination of water soluble and water insoluble components, a bioadhesive agent, and a dispersing agent, is applied to the body cavity, preferably only once, and is retained in the body cavity. At least part, and preferably all of, the solid dosage form is melted, preferably on contact with the body and most preferably from body heat. The formulation is spread substantially uniformly in the body cavity.

The invention also includes a method of treating a viral infection of a body cavity. A solid dosage form, which comprises an antiviral, and a combination of water insoluble and water soluble components is applied to the body cavity, preferably only once, and is retained in the body cavity. At least part, and preferably all of the formulation is melted, preferably on contact with the body and most preferably from body heat. The formulation is spread substantially uniformly in the body cavity.

Another embodiment of the invention includes any combination of the methods of treating a bacterial infection, a fungal infection and a viral infection.

### DETAILED DESCRIPTION OF THE PREFERRED EMBODIMENTS

The present invention is not to be limited by any mechanism described in the specification, because it is defined by the claims.

An embodiment of the invention comprises a solid dosage form in the form of a soft gelatin capsule containing an ointment base. The composition preferably contains active ingredients for treatment of vaginal yeast or fungal infections, including but not limited to those caused by a fungus called Candida. The antifungal may be any antifungal, which is effective to treat oral fungal or vaginal yeast or fungal infections, including but not limited to imidazole derivatives, preferably miconazole nitrate, cyclopirox, clotrimazole, econazole, saperconazole, fenticonazole, sertaconazole, terconazole, itraconazole, ketoconazole, butaconazole, tioconazole, posaconazole, fluconazole, cyclopirox, their pharmaceutically acceptable salts or a combination thereof, and more preferably miconazole nitrate. The antifungal is present preferably in amounts from about 400 mg to about 1200 mg per dose.

Another embodiment of the solid dosage form of this invention may be in the form of a suppository such as that set forth in U.S. Pat. No.4,999,342, which is hereby incorporated herein by reference. Another solid dosage form suppository base may be such as that set forth in U.S. Pat. No. 6,495,157, which is hereby incorporated herein by reference.

The solid dosage form preferably may contain both water insoluble and water soluble components. The water insoluble components may be any water insoluble components, which are acceptable for application to and not unduly irritating to the body cavity, including but not limited to stearyl alcohol, petrolatum, vegetable oil suppository bases or a combination thereof, preferably stearyl alcohol. Water soluble components may be any water soluble components, which are acceptable for application to and not unduly irritating to the body cavity, including but not limited to polyethylene glycols, propylene glycols and glycerin, as well as polysaccharide gums such as carboxymethylcellulose, xanthan gum, other carboxyalkylcellulose products and silica products.

Another embodiment of the invention is the method of treating a fungal infection of a body cavity, preferably in a single dose. The solid dosage forms of this invention may be applied to the body cavity at any time during the day or night, and spread in the body cavity. Upon vigorous exercise or activity, the solid dosage forms of this invention melt quickly and spread throughout the vaginal cavity. At least part, and preferably all of, the solid dosage form is melted, preferably on contact with the body and most preferably from body heat. The method may optionally also include treating a bacterial infection of a body cavity. Another embodiment of the invention is the method of treating a bacterial infection of a body cavity, preferably in a single dose. Solid dosage forms may be applied to the body cavity, and spread in the body cavity. At least part, and preferably all of, the dosage form is melted, preferably on contact with the body and most preferably from body heat. The dosage form is spread preferably substantially uniformly in the body cavity, preferably after the melting of the dosage form has occurred. The dosage form used may be, including but not limited to, any embodiment described above.

Another embodiment of the invention comprises a method of delivering an antifungal, antibacterial, antiviral or any combination thereof ointment (for example those described in the other embodiments) into the vaginal cavity in a gelatin capsule with or without an applicator. Such gelatin capsules are known in the art and are used in connection with products such as Monistat® 1 combination pack (by McNeil-PPC, Inc., Johnson & Johnson, New Jersey). The gelatin capsule may comprise a soft gelatin capsule shell or a two piece hard gelatin capsule shell, preferably a soft gelatin capsule shell. The shell encloses the antifungal, antibacterial, antiviral or any combination thereof ointment (as claimed and taught herein).

In another embodiment of the compositions of this invention, a solid suppository dosage fill mass containing hard fat such as hydrogenated vegetable fat and an active ingredient for treating vaginal infections may be encased within a soft gelatin capsule shell as set forth above, containing gelatin and glycerin. Preferably, said active ingredient is an anti-fungal agent as set forth herein. More preferably, said active ingredient is an imidazole antifungal agent, most preferably, miconazole nitrate. The active ingredient should be present in an amount of from about 2 to about 10% by weight, more preferably, from about 4 to about 8% and most preferably, about 8%. The solid suppository fill mass is an anhydrous composition composed of a hard fat material that melts at or about body temperature (approximately 35° C. to about 39° C.), and can include hard fats such as hydrogenated vegetable oils such as Wecobee FS or Wecobee M (available commercially from The Stepan Company of Northfield, Ill.) or a combination thereof. Preferably, the fill mass is anhydrous and the hydrogenated vegetable fat comprises the remainder of the fill mass in addition to the active ingredient. Preferably, the weight ratio of the combination of fats is approximately 1:1 to about 2:1. More preferably, if a combination of hard fats is utilized, Wecobee M constitutes the minor proportion of the hard fat portion of the composition.

Single dose treatments are most preferable, while multiple doses may also be included in the invention. The formulations of this invention used in such treatments should adhere to the vaginal mucous membrane, not leak or wash out from the vagina, and continue to release the antifungal, antibacterial or both into the vagina for more than 24 hours, preferably for about 72 to about 120 hours, most preferably for at least 70 hours.

Preferably, the solid dosage form utilized in the methods of this invention is a soft gelatin ovule as described in U.S. Pat. No. 6,153,635, which is hereby incorporated herein by reference and is commercially available as MONISTAT®—1 Combination Pack (by McNeil-PPC, Inc., Johnson & Johnson, New Jersey). Additionally, polysorbate 60 may be added to the water soluble components in order to increase the percent of antifungal in the water soluble component. Too little antifungal in the water soluble components will decrease the efficacy of the antifungal or antibacterial, while too much antifungal in the water soluble component will increase its toxicity to the consumer and potential for irritation. The infection is treated at the mucosa and therefore, it is important to retain most of the antifungal or antibacterial at the body cavity mucosa and maintain its concentration for a long period of time, preferably at least 24 hours, more preferably 72 hours and most preferably 120 hours. The preferred embodiment delivers an effective amount of the antifungal, which resides in the vagina for sufficient time after a single dose, to effectively treat the fungal infection without any additional doses.

Further, this embodiment may utilize bioadhesive agents which help to promote adhesion of the ointment to the body cavity mucosa membranes. The bioadhesive agents (including gelling agents and hydrocolloids) may be any bioadhesive agent which is acceptable for application to and not unduly irritating to the body cavity, preferably xanthan gum, sodium carboxymethylcellulose, or mixtures thereof, most preferably a mixture of xanthan gum and sodium carboxymethylcellulose. The fungal infection is located at the body cavity mucous membranes, and the longer residence time of the composition over the prior art promotes the effectiveness of the invention. The bioadhesive agents allow the dosage form to be applied and melted in the vagina, where the dosage form comes into contact with moisture. Then, elements within the dosage form gel and therefore the active antifungal or antibacterial ingredients are retained for sufficient time to effectively treat the infection.

Additionally, this embodiment may include one or more dispersing agents, which may be any dispersing agents acceptable for application to and not unduly irritating to the body cavity, preferably silicon dioxide. Dispersing agents contribute homogenous melt and spread characteristics to the mixture and aids in the adhesion to the body cavity mucous membrane for a controlled release of the antifungal or antibacterial.

Additionally, this embodiment may include one or more antibacterials. The antibacterials may be any antibacterials which. are effective to treat bacterial infections, are acceptable for application to and not unduly irritating to the body cavity. Preferably, the antibacterials are metronidazole, ornidazole, tinidazole, clindamycin, secnidazole, sodium polystyrene sulfate, sodium cellulose sulfate, or mixtures thereof, most preferably metronidazole.

Another embodiment of the invention comprises a solid dosage form for vaginal use comprising one or more antifungals, one or more water insoluble components, one or more water soluble components, and one or more probiotics. The probiotic is preferably probiotic organisms, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbweckii, L. helveticus, L. salivarius, L. collinoides, L. buchneri, L. rogosal, L. bifidum, B. bifidum, B. breve, B. adolescetis or B. longum.

Another embodiment of the invention comprises a solid dosage form for vaginal use comprising one or more antibacterial, one or more water insoluble components, one or more water soluble components, and one or more probiotics. The probiotic is preferably probiotic organisms, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbweckii, L. helveticus, L. salivarius, L. collinoides, L. buchneri, L. rogosal, L. bifidum, B. bifidum, B. breve, B. adolescetis or B. longum.

Another embodiment of the invention comprises a solid. dosage form comprising one or more antifungals, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Another embodiment of the invention comprises a solid dosage form comprising one or more antibacterials, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Probiotics may be incorporated into the embodiment for the establishment and maintenance of the healthy vaginal flora.

Another embodiment of the invention comprises a solid dosage form comprising one or more antifungals, one or more water insoluble components, one or more water soluble components and one or more antivirals. The antiviral may preferably include but is not limited to immunomodulators, more preferably imiquimod, its derivatives, podofilox, podophyllin, interferon. alpha, reticulos, and cidofovir.

Another embodiment of the invention comprises a solid dosage form comprising one or more antibacterials, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Antivirals may be incorporated into the embodiment for treatment of viral infections, including but not limited to genital human papillomavirus (HPV) infections, genital warts, herpes simplex infections and acquired immunodeficiency syndrome (AIDS).

Another embodiment of the invention comprises a solid dosage form comprising one or more antibacterials, one or more water insoluble (or lipophilic) components and one or more water soluble (or hydrophilic) components. The solid dosage form is used to treat body cavity bacterial infections, including but not limited to bacterial vaginosis. The antibacterial may be any antibacterial, which is effective to treat body cavity bacterial infections, including but not limited to metronidazole, secnidazole, sodium polystyrene sulfate, sodium cellulose sulfate or a combination thereof, and more preferably metronidazole. The antibacterial is present in amounts from about 25 mg to about 250 mg per dose.

The water insoluble components may be any water insoluble components, which are acceptable for application to and not unduly irritating to the body cavity, including but not limited to petrolatum, vegetable oil bases or a combination thereof. The water soluble components may be any water soluble components, which are acceptable for application to and not unduly irritating to the body cavity, including but not limited to polyethylene glycols, propylene glycols and glycerin.

The combination of water soluble and water insoluble components are utilized for the base. The water soluble components preferably include components with a mixture of both high and low melting points, more preferably polyethylene glycols, propylene glycols and glycerin. Most preferably, polyethylene glycol 400, which is a liquid, is combined with polyethylene 3350, which is a solid. Additionally, the water insoluble components preferably include components with a mixture of both high and low melting points, more preferably petrolatum and vegetable oils with both high and low melting points.

The following examples are preferred embodiments of the invention.

### EXAMPLE 1

### Clinical Study

A multi-centered, randomized, parallel-group, investigator-blinded study was conducted to compare the safety and efficacy of a solid dosage form containing miconazole nitrate, an antifungal administered at bedtime versus daytime administration. The solid dosage form used in the study was the MONISTAT* 1 ovule product commercially available from Personal Products Company, Skillman, N.J. 08558 in its Combination Pack. The study compared the safety, efficacy and therapeutic cure rate of a single dose of a miconazole nitrate (1200 mg) vaginal OVULE™ Insert, following bedtime or daytime self-administration, for the treatment of vulvovaginal candidiasis. A subject assigned to bedtime self-administration was to remain in bed for at least 30 minutes after dosing. A subject assigned to the daytime regimen self-administered the dose at a subject-determined convenient time during the day within six hours after arising and was informed by the study staff to be active within four hours of insertion of the vaginal insert. The subjects were instructed to apply a small amount of miconazole nitrate (2%) external vulvar cream on the skin outside the vagina as needed, up to twice daily for at least three, days, preferably for five days and up to a maximum of seven days for external symptom relief. The study also determined the symptomatic relief and the mycological and clinical cure rates of bedtime versus daytime administration.

The study population was as follows: 573 subjects were actually enrolled in the study. Subjects were to administer the OVULE™ Insert within 48 hours of the initial admission visit. A post therapy telephone contact was made seven to 10 days following the intravaginal administration of the ovule and a “test of cure” visit was scheduled between 21 and 30 days following intravaginal study drug use to determine therapeutic cure. Of the two treatment groups, at least 20% of subjects assigned to the daytime treatment regimen were to attain a vigorous activity level and 80% were to obtain a moderate or higher activity level for at least one of the four one-hour time intervals following administration of the OVULE™ Insert. The OVULE™ Insert is a soft gelatin shell containing 1200 mg of miconazole nitrate and inactive ingredients including gelatin, glycerin, lecithin, mineral oil, titanium dioxide and white petrolatum. Miconazole nitrate (2%) was the active ingredient of the external vulvar cream and was a formulation identical to MONISTAT*7 Vaginal Cream. Inactive ingredients for the external vulvar cream included benzoic acid, cetyl alcohol, isopropyl myristate, polysorbate 60, potassium hydroxide, propylene glycol, purified water and stearyl alcohol.

A total of 573 subjects were enrolled in the study. 279 subjects were randomly assigned to the Daytime group and 294 subjects were randomly assigned to the bedtime group. There were similar numbers of subjects in the safety and ITT. (“Intent-to-treat”) populations for each group, 278 (99.6%) subjects in the Daytime group and 292 (99.3%) subjects in the Bedtime group. Subjects evaluable for efficacy included 149 (53.4%) in the Daytime group and 163 (55.4%) in the Bedtime group. Three subjects were considered nonevaluable for the safety, ITT or efficacy evaluable populations. The ITT population consisted of all subjects who used either the OVULE* Insert or external vulvar cream.

The results of the study, as set forth in Table I below, show that, in the Daytime group, 86 (57.7%) subjects experienced a therapeutic cure, with 105 (70.5%) subjects and 111 (74.5%) subjects exhibiting mycological and clinical cures, respectively. In the Bedtime group, 83 (50.9%) subjects experienced a therapeutic cure, with 104 (63.8%) subjects and 120 (73.6%) subjects exhibiting mycological and clinical cures, respectively. There was, surprisingly, no statistically significant difference between groups, as set forth in Table I below. It would have been expected, prior to applicants' invention, that administering solid dosage forms at any time during the day while patients are upright and mobile would have resulted in loss of the dosage form due to the action of gravity and movement.

TABLE I

| | Response/Group | | | |
|---|---|---|---|---|
| | Daytime | | Bedtime | |
| Therapeutic Cure Rate | 86/149 | 57.7% | 83/163 | 50.9% |
| Mycological Cure Rate | 105/149 | 70.5% | 104/163 | 63.8% |
| Clinical Cure Rate | 111/149 | 74.5% | 120/163 | 73.6% |

Table II below illustrates the therapeutic, mycological and clinical cure rates at the test-of-cure visit according to activity level for efficacy evaluable subjects randomized to the Daytime group. Subjects were classified as having a high activity level if, at any of the four hourly evaluations following study drug administration, the activity level was moderate or vigorous; all other subjects were classified as having a low activity level. Of the 43 subjects classified as having a low activity level, 26 (60.5%) subjects were considered a therapeutic cure, 35 (81.4%) subjects were considered a mycological cure, and 30 (69.8%) subjects were considered a clinical cure. Of the 105 subjects classified as having a high activity level, 59 (56.2%) subjects were considered a therapeutic cure, 69 (65.7%) subjects were considered a mycological cure, and 80 (76.2%) subjects were considered a clinical cure. When compared to Table I, therapeutic, mycological and clinical cure rates for subjects with a high activity level were slightly higher relative to cure rates of subjects in the bedtime group. There were no statistically significant difference in the therapeutic cure rates between activity levels.

TABLE II

| Response/Group | |
|---|---|
| Low Level | High Level |

| | | | | |
|---|---|---|---|---|
| Therapeutic Cure Rate | 26/43 | 60.5% | 59/105 | 56.2% |
| Mycological Cure Rate | 35/43 | 81.4% | 69/105 | 65.7% |
| Clinical Cure Rate | 30/43 | 69.8% | 80/105 | 76.2% |

In both the Daytime and Bedtime groups, for the subjects who reported itching symptoms initially, the median time to relief of itching was 36 hours. Again, surprisingly, there was no statistically significant difference between groups. For the subjects who reported burning symptoms, the median time to relief of burning was 24 hours in the Daytime group and 36 hours in the Bedtime group. Surprisingly, there was no statistically significant difference between groups. Median time to relief for the symptom of irritation in the Daytime group was 48 hours; the time to relief for irritation in the Bedtime group was 42 hours. Again, surprisingly, there was no statistically significant difference between groups.

### EXAMPLE 2

### Distribution and Retention of Miconazole Nitrate

The publication, “Vaginal Distribution of Miconazole Nitrate Suspension from Administration of a Single Vaginal Insert,” (Barnhart, et al.), J. Reprod. Med. 49:83-88, 2004, which is hereby incorporated herein by reference, describes the distribution patterns and retention of miconazole nitrate in the. vagina. We have found that administration of a soft gelatin insert during the day, followed by vigorous exercise, appears to distribute the drug contained therein throughout the vagina more readily. We believe that such distribution should lead to a better cure rate than merely having the patient insert the product and go to sleep.

It is understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

What is claimed is:

1. A method of treating a vaginal infection in a patient comprising inserting a solid dosage form comprising an anti-fungal or anti-bacterial effective amount of an anti-fungal or anti-bacterial active ingredient into the vagina at any time after the patient awakens until thirty minutes prior to the patient retiring for the night wherein said solid dosage form comprises a solid dosage form comprising hard fats that melt at approximately body temperature, said solid dosage form being surrounded by a solid gelatin capsule.

2. A method according to claim 3 wherein said solid dosage form comprises a suppository comprising hydrogenated vegetable fat.

3. A method according to claim 1 wherein said anti-fungal active ingredient is miconazole nitrate.

4. A method according to claim 1 wherein said anti-bacterial active ingredient is metronidazole.
